(19) United States
(12) Patent Application Publication
(10) Pub. No.: US 2008/0220494 A1
(43) Pub. Date: Sep. 11, 2008

(54) Method of transforming extracellular components of the dermis into a gelatinized substance

(76) Inventors: Alberto Nazareno Bolgiani, Buenos Aires (AR); Norma Lidia Bustos, Buenos Aires (AR)

Correspondence Address: DEFILLO & ASSOCIATES, INC., P.O. Box 14104, Clearwater, FL, 33766, US

(21) Appl. No.: 12/074,933

(22) Filed: Mar. 7, 2008

Related U.S. Application Data
(60) Provisional application No. 60/893,732, filed on Mar. 8, 2007.

(51) Int. Cl. C12N 13/00

(52) U.S. Cl. 435/173.9

(57) ABSTRACT

The present invention relates to a method to transform all extracellular components of a dermis into a gelatinized substance with long permanence, wherein these extracellular components are obtained from autologous samples of skin of a patient. This method eliminates epidermic cells completely and allows obtaining autologous dermis. The extracellular components of the autologous dermis are obtained by the lysis of collagen fibers. Furthermore, the dermis is exposed to gamma-ray irradiation to eliminate all living cells.

### REFERENCE TO RELATED APPLICATIONS

This application is a non-provisional of Provisional Application No. 60/893,732 filed Mar. 8, 2007.

### FIELD OF THE INVENTION

The present invention relates to cosmetic dermatology. Specifically, the invention provides a new method to purify the extracellular components of the dermis arising from autologous samples of skin.

### BACKGROUND OF THE INVENTION

In the last few years, several injectable materials and fillers have been used in cosmetic dermatology for the treatment of facial lines, atrophic lips and skin aging. The naso-labial folds, the glabellar area, the wrinkles called crow's feet and the lips are most often injected.

Several different types of products have been utilized. Some fillers needs double skin testing before injecting, others are not theoretically allergenic and according to companies do not require any test. However, several adverse events have been documented. With the trend toward less invasive procedures, often in younger patient population, there is an increasing patient demand for the benefits of soft-tissue injectable fillers skin materials.

Given the numerous options that now exist and the different chemical compositions and potential biological behavior of the various materials, it is imperative that plastic surgeons be knowledgeable about contemporary injectable fillers. It is also important to understand that although all of the known and normally used injectable fillers are safe, the concepts of their long-term volume persistence and how they compare with each other largely remain anecdotal with few prospective controlled clinical trials and subsequent U.S. Food and Drug Administration, U.S. FDA, approval of them usually does not include lip augmentation, even though it is one of the more commonly injected facial sites.

Given that our skin is a complex engineered covering, to get a better understanding it is necessary to know the anatomy and physiology of the skin. The skin has a slightly acidic coating of oil at the surface. This coating protects the skin against some bacteria. Below the surface is a complex of sweat and oil glands hair follicles, blood vessels, nerves and muscle tissue. These are held together by a tough connective tissue called collagen.

Collagen is very important in determining the health of the skin. The relative health of the collagen determines the contour of the skin, how wrinkled and lined it is. Healthy collagen is often called soluble collagen, because it can absorb and hold moisture. Below the collagen is a layer of fat and muscle, which provides some contour and acts as a cushion and as insulation.

The skin has two main layers. The inner layer is known as dermis, and the outer layer is known as epidermis. The various glands such as the oil and sweat glands originate in the lower dermis. From here, they rise to the surface of the skin to eliminate waste matter.

Lower dermis also acts as a cushion for the rest of the skin. It contains the finely distributed muscles of the skin which regulate body temperature. The dermis is the layer that lies underneath the epidermis. It consists of bundles of tough fibers which give the skin its elasticity, firmness and strength. There are also blood vessels, which feed vital nutrients to these areas. Dermis determines the tone of the skin. The epidermis is top layer of skin and the one we can actually see. It protects our body from invasion and infection and help to seal in moisture. It's built up of several layers of living cells which are the topped by sheets of dead cells. It's constantly growing, with new cells being produced at its base. They quickly die, and are pushed up to the surface by the arrival of new ones. These dead cells eventually flake away, which means that with every new layer of skin is another chance to have a soft, glowing complexion.

The epidermis is responsible for coloring, as it holds the skin pigment. It ranges in thickness from 1/20-th of an inch on the palms and soles, to 1/200-th of an inch on the face.

With regard to injectable materials and fillers that have commonly been used in cosmetic dermatology for treatment of facial lines, atrophic lips and skin aging fillers, those can be:

Autologous meaning like one's own dermis or fats. These are, especially, used in deep depressions that need subcutaneous filling like the nasogenians furrows; or

Heterologous meaning obtained of animal products or synthesis. These are divided in:

a) Biodegradable meaning non-synthetic products that the human organism is able “to degrade”, disappearing totally in the body in the variable term after its application. The most used are bovine collagen, hialuronic acid and polilactic acid. They display the disadvantage of the necessity to apply periodic injections to maintain the results, and that it is necessary to perform skin testing before injections. Moreover a new collagen source from cadavers is now on the market, but a long-term follow-up is necessary to know its efficacy and safety.

b) Non-biodegradable meaning synthetic products. Its application is considered definitive. The most used for years, while at the moment prohibited in many countries, has been liquid silicone. At the moment methacrylate crystals are used. Its greater disadvantage is that, if by any error of technique, or an excess of volume or any anomalous reaction of tissues, the result is not what was hoped for by a patient. This product cannot be extracted.

Ideally, an injectable implant should have a lack of any significant inflammatory response (be highly biocompatible), be easily introduced into the recipient site by injection (have a good flow behavior through a small-gauge needle), and produce an acceptably long period of volume retention (i.e., month to years). Each current U.S. FDA approved filler and those under U.S. FDA application/study exhibit differences in these three basic characteristics.

We also can find products of synthetic origin, like those that contain calcium hydroxyapatite, a natural substance that is present in bones and teeth. It is mainly used for the reconstruction of bones. In cases that is injected, natural collagen form on the hydroxyapatite and give a fullness appearance.

In regard to non-synthetic products, which are the preferred ones, the first widely used injectable fillers continues to decline, remains the standard to which all other injectable fillers continue to be compared. Its animal derivation and shot longevity have led to its decrease in popularity. In U.S. FDA clinical trials, however, its long history of clinical use and data make it the frequently used control. These are derivates of bovine collagen. These products consist of 96 percent type I collagen, with the remainder being type III collagen. It is 3.5 percent bovine dermal collagen by eight, suspended in phosphate-buffered physiologic saline. A variant thereof consists of 3.5 percent bovine dermal collagen cross-linked by grutaraldehyde, which makes it more resistant to biodegradation becoming more viscous but less immunogenic than the first one. The clinical characteristics of these bovine collagen products are that they are injected in the papillary dermis and are U.S. FDA approved for fine lines and shallow acne scars and for moderate lines and deeper acne scars. Their results typically last from 2 to 3 months.

Although these bovine collagen derivates are known to be safe, with very few local complications, with a clinical effectiveness and versatility well established and coming diluted with 0.3% lidocaine, which may reduce pain on injection, they have some disadvantages. Because as they are bovine derivates, they require skin testing and thus should not be used on the day of consultation, unless it is an established patient. Of note, 3 percent of patients develop a sensitivity reaction even with a normal skin test. Local adverse effects include erythema, induration, pruritus, and skin decoloration. Furthermore, a systemic hypersensitivity reaction can occur 48 to 72 hours after injection. This is manifested by fever, malaise, and urticaria, which are treated with short-term oral steroids. Granulomas have also been reported. Reactivation of herpes is possible with lips injections; thus, patients with a positive history need antiviral prophylaxis. Other complications include necrosis of the overlying skin and unilateral vision loss caused by retinal artery occlusion. They are also contraindicated in patients with lidocaine allergies. The material starts to be degraded immediately after injection, with clinical effects observable for a few months. The reduction in injection volume is essentially linear over time.

More recently non-synthetic injectable fillers have arisen in an eventual response to concerns and problems with bovine derived collagen. These are the human-based collagen homologues. These offer the promise of decreased immunogenicity and longer lasting results. Those products are injectable implants derived from highly purified human collagen approved for use in facial aesthetic surgery in March of 2003 and are the only U.S. FDA approved dermal fillers made from human collagen. The collagen is cultured from a single cell line of human dermal fibroblast that have been used for over 10 years to manufacture human-based tissues; these cells produce natural collagen that is then isolated and purified for injection. The cell line undergoes extensive testing for viruses, retroviruses and tumorigenicity. This product should be refrigerated but must not be frozen and the U.S. FDA approved it for shallow wrinkles or acne scars and it is injected into the superficial papillary dermis and for the correction of more pronounced wrinkles or scars. Results typically last from 3 to 6 months.

The Inamed Human Collagen Immunogenecity Clinical Study demonstrated that the 95 percent upper confidence interval for experiencing a hypersensitivity reaction against those products is less than 1.3 percent. This is less than the incidence of immunologicaly related adverse events reported with bovine collagen in treated patients who initially had a negative skin test. Unlike bovine collagen implants, these dermal fillers do not require a pretreatment skin test before application. This has been established thought their preapproval in U.S FDA application study. Thus patients may undergo treatment at the time of their initial consultation they come diluted in 0.3% lidocaine, which may have some benefit in reducing pain on injection. However, the use of these products is contraindicated in patient with a known allergy to lidocaine. The longevity of this material appears to be similar to bovine collagen.

There also exists a novel non-synthetic product having many indications in reconstructive and aesthetic surgery. It consists of injectable decellularized processed dermal allografts that were U.S. FDA approved in 2000. The material is originally obtained from tissue banks compliant with the guidelines of the America Association of Tissue Banks and the U.S. FDA. First, the epidermis is completely removed, followed by removal of dermal cells and stabilization of the dermal matrix through inhibition by metalloproteinase. The material is then cryofractured, breaking down the acellular dermal matrix into micronized particles that are packaged into syringes. At the time of clinical use, it is necessary to hydrate this powder. This product must be reconstituted with 1 cc of 1% lidocaine and is injected into a subcutaneous space. It has more viscous consistency after hydration and requires a large caliber needle for introduction. It is indicated for use in nasolabial folds, lips and acne and depressed scars having no immune response because cells exhibiting major histocompatability complexes I and II have been removed. Moreover patients can be treated at initial consultation because no skin testing is needed.

Results typically last from 3 to 9 months. On the other hand, it has a very important disadvantage. Because of its large particle size it yields less smooth results than most other implants and it can be more painful because of the size of the needle. Patient should also be made aware of postinjection edema. Furthermore, its use should be avoided in periocular line correction and glabellar contouring to avoid the risk of intravascular injection and migration. This product is supplied in an antibiotic-supplemented medium. Thus, patients with sensitivities to the antibiotic should not received this material. The size of the hydrated particles and the needle do not allow intradermal injection, limiting its use to a subcutaneous space. Moreover, there are no clinical trials that demonstrate its longevity to be superior to other collagen-based implants.

Tissue Banks collagen material, other than from the dermis, is available from numerous other sources. There exists a very tight fibrillar collagen weave, offering a potentially more dense form of collagen implant, that comes from human cadavers, and may be obtained from the American Association of Tissue Banks guideline-compliant tissue bank. Preserved fascia lata has been used for a long time as a sheet graft material, but has only recently become available in injectable form. Clinical Characteristics Fascian is processed into particle sizes of 0.25, 0.5, 1.0, or 2 mm, of which 80 mg of volume is inserted into each syringe. This material may be stored at room temperature for years.

Clinically, Fascian is indicated for deep wrinkles, scars, fat atrophy, and prominent nasolabial folds. At the time of injection, the fascia particles are initially hydrated in 3 to 5 cc of 0.3% lidocaine solution. This produces a thick paste that can be extruded through a largebore needle. The duration of results last from 6 to 8 month, but other anecdotal reports show duration of 3 to 6 months.

Given that this collagen origin is not from the same patient, it can display adverse reactions. Thus, a previous test of tolerance is required. Furthermore, trace amount of polymyxin B sulfate, bacitracin and gentamicin are present in those types of implants. So, patients with known allergies to these antibiotics should avoid them. The large size of the needle needed for introduction results in the potential for increased bruising. In addition, local anesthesia infiltration into the recipient or nerve blocks may be needed for patient comfort during the procedure. Documentation of the longevity of the material has not been reported in any prospective or controlled patient series.

As seen above, until the moment, there is no single injectable filler that has all of the desired characteristics. Understanding the advantages and disadvantages of one filler over another, with an appreciation of their limitations, is extremely helpful in achieving the patient's goals and minimizing the risk of potential adverse reactions.

Autologous collagen seems to be the best approach as a dermatological injectable filler. Since it is prepared from an autologous sample, it has no risk of hypersensitivity reaction, and infections are the only risks. Many efforts have been made in the last few years to find a better and more rapid method to obtain the extracellular components of the dermis, but a major drawback is the need to prepare it before injecting.

### SUMMARY OF THE INVENTION

It is therefore an object of the present invention to provide a rapid method of skin sample processing that allows transforming the extracellular components of the dermis into a gelatinized substance.

It is still another object of the present invention to provide a method of skin processing that allows obtaining all the extracellular components of the dermis.

It is still another object of the present invention to provide a method of skin processing comprising the steps of: separating the dermis from the epidermis, homogenizing the separated dermis to obtain a final product, and irradiating the final product.

It is another object of the invention to provide a method to obtain a gelatinized injectable substance comprising all the extracellular components of the dermis.

It is still another object of the present invention to provide a method able to eliminate the epidermic cells completely. It is a further object of the present invention to provide a method which allows obtaining autologous dermis.

It is a further object of the present invention to provide a method which allows homogenization of the autologous dermis by means of lysis of collagen fibers.

It is still another object of the invention to provide an autologous gelatinized substance which may be applied by intradermic injection without the necessity to use anesthesia.

It is a further object of the present invention to provide an autologous gelatinized substance which has long permanence.

It is another object of the present invention to provide an autologous gelatinized substance that, once injected, does not produce any type of adverse inflammatory reaction nor rejection.

It is still another object of the invention to provide an autologous gelatinized substance which is easily introduced into the recipient site by injection, having good flow behavior through a small gauge needle.

These and other objects and advantages of the invention will be better understood from the following detailed description of preferred embodiments of the inventive process.

### DETAILED DESCRIPTION OF PREFERRED EMBODIMENTS

Now referring in detail to the invention, the same refers to a new method of skin processing that allows obtaining all the extracellular components of the dermis arising from autologous samples of skin, where “autologous” means “a patient's own”. The obtained gelatinized substance has many present possible uses, and surely more will be added as its use expands. The present uses are mainly for correction of: depressions by acne scars; depressed scars; wrinkles of all types and locations; nasolabial folds; stuffing in the lips; depressions by cellulite; stuffing of the nose; and modifications of the corporal contour.

Now referring to the procedure, once the dermoepidermic tissue (skin sample) is extracted from the patient, the same is transferred to the Laboratory in a culture medium where it is processed. It is important to notice that it is possible to use skin samples of possible previous operations, as skin of previous aesthetic surgeries (eyelids, lifting) and also from other surgeries, not specifically aesthetic ones, as from, for example, a Caesarean one. That is, that a skin sample of any zone of the body can be used for this purpose.

It is important to clarify that it is not necessary, however, that the skin sample is stored from the past. Simply the advantage of a previous surgery, often to very early ages, allows counting on young dermis to be used when the normal process of aging requires it, or when a fortuitous circumstance makes necessary an aesthetic correction. But, if a patient does not have this stored material, an aesthetic surgeon can obtain a piece of skin from a nonvisible zone to be used in the manufacture of his own filling material.

Next, the processing is carried out in a laboratory in a camera with a laminar flow under strict norms of security (sterility) and it consists of the following stages: separating the dermis from the epidermis, homogenizing the separated dermis to obtain a final product, and irradiating the final product. Separating the dermis from the epidermis stage consists of: taking the skin pieces with sterile tweezers and placing them in a sterile Petri dish; adding 20 milliliters of sterile physiologic solution with a 25 millilitre pipette; transferring the pieces of skin to another Petri dish, by means of a scalpel, a pair of scissors

and dissection tweezers; eliminating the rest of the fat and hairs; cutting the skin in pieces of approximately 1 cm² with a scalpel; putting them in the Petri dish without superposing the skin pieces; adding, by means of a 25 milliliter pipette, 20 milliliters of a dispase solution to each Petri dish; incubating in a heater (37° C.) during 24 hours; by means of dissection tweezers, separating the dermis from the epidermis; discarding the epidermis; transferring with tweezers the pieces of dermis to a 50 milliliter centrifuge tube; adding, by means of a 25 milliliter pipette, 20 milliliters of the culture medium DMEMc (DMEM Gibco Cat N^{o} 11995-065) with 10% fetal bovine serum (BFS) to neutralize the effect of dispase; shaking it manually; centrifuging in a range of at least 1100 rpm and at most 1300 rpm for a period of at least 4 minutes and at most 6 minutes, more preferably at 1200 rpm for 5 minutes; discarding a supernatant, using a 10 milliliter pipette; repeating the washing step for three times or until the supernatant is colourless; discarding the supernatant, using a 10 milliliter pipette; adding with a 10 millilitre pipette 5 milliliters of physiological solution; and taking it down to a temperature lower than −60° C. to freeze it.

Homogenization of the autologous dermis stage allows lysis of collagen fibers, and it consists of: defrosting in a 37° C. water bath; placing the fragments into 15 milliliters conical tubes; homogenizing during a period of time of at least 18 seconds and at most 22 seconds centrifuging at least 9000 rpm and at most 11000 rpm using a tissue homogenizer, more preferably during 20 seconds at 10000 rpm; waiting for 5 minutes to let the bottle go down; homogenizing for at least 8 seconds and at most 12 seconds at at least 18000 rpm and at most 22000 rpm, more preferably during 10 seconds at 20000 rpm; introducing some sterile tweezers inside the bottle to verify that there are not fragments (fibers) that have not been lysed (if the obtained suspension is not homogeneous, it is recommended to repeat this process); centrifuging at least 1000 rpm and at most 1400 rpm for a period of time of at least 8 minutes and at most 12 minutes, more preferably at 1200 rpm for 10 minutes; discarding fat by pipette extraction; placing the homogenized dermis into a sterile Petri dish; absorbing with a syringe and needle, putting 1 millilitre in each syringe or according to a request of a medical doctor; replacing the used needle by the definitive one with its protection cover; pocketing each syringe in a double polyethylene bag, properly labeled; and taking it down to a temperature lower than −60° C. to freeze it. The irradiation stage must be carried out in an irradiation plant with a cobalt 60 source, for inactivation of living cells, and this step consists of: irradiating a sample in a box containing dry ice, without defrosting it, with gamma-rays at a recommended sterilization dose of at least 20 kGy and at most 30 kGy, more preferably at an irradiation dose of 25 kGy, returning the sample to a temperature smaller than −60° C., until the moment of its use.

The unit, Gy, is a unit of deposited radiation energy or absorbed dose called Gray, and is equal to a Joule of radiation energy absorbed by a kg of matter. Alternatively, other methods of sterilization are used including exposure to ultraviolet light, x-rays, chemical and anti-biotic treatment.

The final product contains all extracellular components of dermis: collagen fibers; proteoglicans; elastine; hialuronic acid; chondroitin sulphate; and heparan sulphate; all of those in the same proportions in which they are found in normal dermis. Furthermore, the final autologous product does not contain any living cellular component, because those cellular components like queratinocytes, melanocytes, fibroblasts, Langerhans cells and Merckel cells are eliminated to avoid inflammatory formation of epidermic inclusions, inflammatory reactions and possible suppurations.

A great advantage of this method is that it is not necessary to use the product immediately after it is obtained; indeed the obtained injectable substance can be stored for conservation for future use by cryopreservation. With this method, the product can be conserved for an infinite time without losing its structure.

The same one, after processing and cryopreservation, will be able to be applied, when it is required for different treatments without producing any inflammatory reaction or rejection.

Another important advantage of this final product is that, unlike other products, it does not contain any added substance, being absolutely natural, it is given to the surgeon ready for its application, and it does not require any previous procedure prior to its injection. It is important to notice that the dermis, once extracted from the patient, does not undergo the process of aging, because it is processed and then maintained in a state of freezing at low temperature (cryopreservation). Those low temperatures do not permit the dermal components to deteriorate over the passage of time. That means that, if at some moment of a person's youth, a skin sample is extracted to be put under this method of processing and the final product is later injected (20 or 30 years later) into this person, he/she is injecting his/her own dermis, but 20 or 30 years younger.

Regarding the way of action, once injected it acts like structure. So that a patient's own cells invade this patient's own gelatinized injected substance, secreting more extracellular substance, serving as filling, and maintaining the filling effect for a long term. An advantage of this product is that the substance does not produce any allergic reaction, it is not rejected nor degraded because it contains 90% of the patient's own extracellular dermal components.

Moreover, once the substance has been applied, the doctor can model the filling very easily allowing him to give to the dermis the format that is required, obtaining a filling that has a long permanence. Even when there is no long term pursuit, there are works that demonstrate that the dermis has a faster vascularization than any other injected autologous soft tissue, and therefore it remains for more time without being reabsorbed. When acting like a structure that provides scaffolding for cellular invasion, these cells, suitably nourished, regenerate the skin structure in a complete way.

With this product it is not necessary to make allergic reaction tests prior to injection, because it contains a part of the patient's own tissue without any added element.

Moreover, it does not produce inflammatory processes given that, in addition to being a patient's own tissue, it does not contain any elements that can induce inflammatory processes (like follicles, sebaceous glands, nor sweat glands).

This method makes unnecessary the use of solvents for its injection, thus non-autologous substances are not added. This method provides a safe product, of the patient's own body, without additives and with all the structural components of the dermis.

Ideally, an injectable implant should have a lack of any significant inflammatory response (be highly biocompatible), be easily introduced into a recipient site by injection (have good flow behavior through a small-gauge needle), and produce an acceptably long period of volume retention (i.e., months to years). The method of the present invention allows the obtaining of a final gelatinized substance that has all of these three optimum conditions. Moreover, its injection does not require the use of lidocaine, or any other anaesthesia.

While there is described herein certain specific method and process steps embodying the present invention, it will be manifest to those skilled in the art that modifications may be made without departing from the spirit and the scope of the underlying inventive concept. Therefore, the present invention shall not be limited to the particular processes herein described except by the scope of the appended claims.

What is claimed is:

1. A method of transforming extracellular components of a dermis obtained from autologous skin samples of a patient into a gelatinized substance, the method comprising the steps of:
separating said dermis from epidermis of said samples of skin;
homogenizing said dermis to obtain a final product; and
irradiating said final product.

2. The method of claim 1, wherein the step of separating said dermis further comprises:
cleaning said skin;
separating under proteolysis said dermis from said epidermis;
washing said dermis; and
freezing said dermis.

3. The method of claim 2, wherein the step of cleaning said skin further comprises:
selecting skin pieces;
adding sterile physiologic solution to said skin pieces;
eliminating fat and hairs from said skin pieces; and
cutting said skin pieces into smaller skin pieces.

4. The method of claim 2, wherein the step of separating under proteolysis said dermis from said epidermis further comprises:
adding a dispase solution to said skin;
incubating said skin in said dispase solution in a heater during 24 hours;
separating said dermis from said epidermis;
discarding said epidermis; and
neutralizing an effect of said dispase.

5. The method of claim 4, wherein the step of neutralizing an effect of dispase is made by an addition of culture medium supplied with fetal bovine serum to said dermis, which has been separated from said epidermis.

6. The method of claim 2, wherein the step of washing further comprises:
centrifuging said dermis, which has been separated from said epidermis;
discarding a supernatant; adding a physiological solution; and
shaking manually said dermis in said physiological solution, and
the step of separating under proteolysis said dermis from said epidermis further comprises eliminating said epidermic cells completely.

7. The method of claim 6, wherein the centrifuging step is carried out in at least 1100 rpm and at most 1300 rpm for a period of at least 4 minutes and at most 6 minutes.

8. The method of claim 6, wherein the step of washing is repeated at least three times until the supernatant is colorless.

9. The method of claim 1, wherein the step of homogenizing said dermis further comprises:
washing said dermis;
defrosting said dermis;
placing said dermis into conical tubes;
subjecting said dermis to a first homogenizing step during a period of time of at least 18 seconds and at most 22 seconds in at least 9000 rpm and at most 11000 rpm using a tissue homogenizer;
letting said dermis rest for a waiting period;
subjecting said dermis to a second homogenizing step for a period of time of at least 8 seconds and at most 12 seconds in at least 18000 rpm and at most 22000 rpm to form a homogenized dermis;
centrifuging said homogenized dermis in at least 1000 rpm and at most 1400 rpm for a period of time of at least 8 minutes and at most 12 minutes to form a centrifuged dermis;
discarding fat from said centrifuged dermis;
absorbing said centrifuged dermis into a syringe and needle; pocketing said syringe in double polyethylene; and
freezing said syringe.

10. The method of claim 1, wherein the step of homogenizing comprises lysing collagen fibers.

11. The method of claim 9, wherein the defrosting step is carried out in a 37° C. water bath, and the first homogenizing step is carried out for 20 seconds at 10000 rpm, and the second homogenizing step is carried out for 10 seconds at 20000 rpm, and the freezing step is carried out at a temperature of at most minus 40 degrees C. and at least minus 100 degrees C.

12. The method of claim 2, wherein the freezing step is carried out at a temperature of at most minus 40° C. and at least minus 100° C.

13. The method of claim 1, wherein the step of irradiating further comprises the steps of: irradiating said samples of skin with gamma-rays in a sterilization dose of at least 20 kGy and at most 30 kGy, and freezing said samples of skin.

14. The method of claim 13, wherein the step of irradiating is carried out with said product in dry ice and further comprises inactivating all living cells with a sterilization dose of 25 kGy.

15. The method of claim 14, wherein said living cells are cells selected from the group consisting of autologous cells, microorganism cells, and a combination of autologous cells together with microorganism cells.

16. The method of claim 9, wherein the step of centrifuging is made at 1200 rpm for 10 minutes.

17. A process of manufacturing a gelatinized substance obtained from a dermis comprising the steps of: isolating all autologous extracellular dermal components; and forming a gelatinized product from said components by homogenizing said components.

18. The process of claim 17, wherein said extracellular components of said Dermis comprise: collagen fibers, proteoglicans, elastine, hyaluronic acid, chondroitin sulphate, and heparan sulphate.

19. The process of claim 18, wherein said extracellular components of said Dermis are present in the same proportions that are present in in-vivo autologous dermis.

20. The process of claim 19, wherein the step of isolating further comprises Sterilizing said components such that said product is free of any living cells, and the step of forming a gelatinized product further comprises freezing said product and conserving said product in a frozen state free of temporal limitation until said product is to be put into a patient, said product is injectable into a patient with normal neurological sensation and free of anesthesia.
